(19) United States
(12) Patent Application Publication
(10) Pub. No.: US 2008/0140037 A1
(43) Pub. Date: Jun. 12, 2008

(54) Crosslinker for Superabsorbent Polymers

(76) Inventor: Thomas H. Newman, Midland, MI (US)

Correspondence Address: SMITH MOORE LLP, P.O. BOX 21927, GREENSBORO, NC, 27420, US

(21) Appl. No.: 11/720,554

(22) PCT Filed: Oct. 26, 2005
(86) PCT No.: PCT/US05/38731

§ 371 Date: Jul. 5, 2007

Related U.S. Application Data
(60) Provisional application No. 60/633,043, filed on Dec. 3, 2004.

(51) Int. Cl. A61F 13/49; C08F 20/10; C07D 307/16; C07C 69/593; C08J 3/24

(52) U.S. Cl. 604/367; 525/330.3; 525/386; 549/499; 560/190

(57) ABSTRACT

The crosslinker of the invention is an asymmetrical polyvinyl crosslinker that disassociates at elevated temperature, and is especially useful in the preparation of superabsorbent polymers.

### BACKGROUND OF THE INVENTION

The invention relates to compounds that are useful as crosslinkers in the manufacture of water-swellable, water-insoluble polymers.

Superabsorbent polymers are well-known materials that commonly are used in items such as cable wrap, agricultural products, packaging, and personal care articles such as diapers. These polymers are known to absorb several times their weight of, for example, water, saline solution, urine, blood, and serous bodily fluids.

In the manufacture of such polymers, it is desirable to produce a hydrogel having a high crosslink density while in the polymerization reactor, as this provides a hydrogel that is easy to process. It is known that absorption capacity is inversely proportional to cross-link density, i.e. a hydrogel with the desired high crosslink density will have a low absorption capacity. However, the manufacturers of absorbent articles and devices favor a final polymer product that has a high absorption capacity, i.e. a low crosslink density. Thus, the manufacturers of superabsorbent polymers are faced with a dilemma.

One possible solution would be to simply heat the hydrogel sufficiently to break down enough of the cross-links to produce a product with the desired low crosslink density. Unfortunately, for the cross-linkers in commercial use, heating the polymer sufficiently to accomplish that goal in an acceptable time period would lead to an undesirable degree of polymer decomposition. Another potential way to achieve low crosslink density is to simply use a low level of crosslinker; however, this leads to gels that are difficult to process in the reactor and an undesirable increase in uncross-linked polymer.

WO 02/04548 A1 discloses the use of certain thermally unstable crosslinkers in the production of solid polymeric coatings, but is silent regarding the use of such materials for manufacturing superabsorbent polymers.

It would be desirable to have a crosslinker for the production of superabsorbent polymers that would allow the preparation of a high capacity final product from a hydrogel having a high crosslink density, while at the same time not increasing the amount of volatiles released upon heating of the hydrogel. It would also be desirable to have a crosslinker that could accomplish this goal without requiring surface crosslinking of the polymer.

### SUMMARY OF THE INVENTION

The present invention includes such a crosslinker composition comprising a compound of at least one of the following formulas:

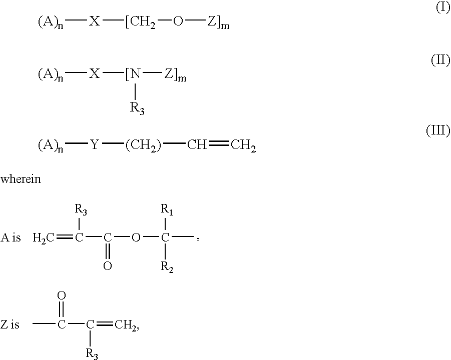

X is an aromatic moiety, an aliphatic moiety, or a mixture thereof, Y is O, N, an aliphatic moiety that may contain one or more O or N atoms, or a mixture thereof, n is from 1 to about 3, m is from 1 to about 3, R₁ and R₂ are independently C₁ to C₄ alkyl, and each R₃ is independently H or methyl.

The invention further includes superabsorbent polymers prepared using the crosslinker composition of the invention, and the use of the crosslinker composition to prepare such polymers.

Surprisingly, the crosslinker of the invention allows one to have a highly-cross-linked, easily-processed hydrogel in the polymerization reactor, which crosslinker is controllably degradable upon heating and which can result in a product of desirably lower crosslink density, and higher absorption capacity, than the gel in the reactor. Another notable advantage of the crosslinker of the invention is that it typically does not evolve volatile fragments of the crosslinker molecule as the polymer degrades.

### DETAILED DESCRIPTION OF THE INVENTION

The crosslinker is represented by one of the three formulas shown hereinabove. The crosslinker of the invention is an asymmetrical polyvinyl crosslinker that disassociates at elevated temperature. A common feature of these compounds is that they contain at least 2 ethylenically unsaturated moieties, and are readily polymerizable in a reaction mixture used to prepare a superabsorbent polymer. Furthermore, at least one of the ethylenically unsaturated moieties is a tertiary ester of (meth)acrylic acid. There is also at least one other polymerizable ethylenically unsaturated moiety, such as a primary ester of (meth)acrylic acid, an amide of (meth)acrylic acid, or an allylic moiety.

Preferably, Y is —CH₂—, or a 5-membered heterocyclic ring containing one oxygen atom and 4 carbon atoms and having a pendant methyl group. R₁ and R₂ are preferably methyl. X is preferably —CH₂—. The most preferred embodiment is the compound 3-methyl-1,3-butanediol diacrylate. Mixtures of the inventive crosslinkers can be employed.

The crosslinker of the invention can be prepared using common organic chemistry procedures that are well known to those skilled in the art. For example, 3-methyl-1,3-butanediol diacrylate can be synthesized from the reaction of acryloyl chloride and 3-methyl-1,3-butanediol utilizing an amine, such as triethylamine, to scavenge the evolved hydrogen chloride. In one embodiment of the invention, the crosslinker is prepared via a coupling reaction using an acryloyl chloride or a methacyloyl chloride and a co-reactant that is a diol (I^{†}), an amino-alcohol (II^{†}), or an allyl alcohol (III^{†}) of the following formulas:

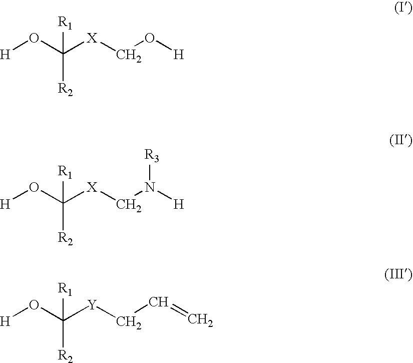

wherein X is an aromatic moiety, an aliphatic moiety, or a mixture thereof, Y is O, N, an aliphatic moiety that may contain one or more O or N atoms, or a mixture thereof, R₁ and R₂ are independently C₁ to C₄ alkyl, and R₃ is independently H or methyl. The stoichiometric amounts of acryloyl chloride or methacryloyl chloride to reagents (I^{†}), (II^{†}), or (III^{†}) are 2:1, 2:1, and 1:1, respectively, and amounts less than or greater than the stoichiometric amounts can be employed. However, it is preferred to employ an excess of the acryloyl chloride or methacryloyl chloride in order to maximize the yield of the desired crosslinker. The amount of excess can determined empirically, based on the purity of the reagents, solvents and other conditions, as is known by those skilled in the art. Suitably, the amount employed in excess of the stoichiometric amount is on the order of from about 10% to about 1,000%, based on the weight of the co-reactant, and more preferably is from about 20% to about 300%. Acryloyl chloride and methacryloyl chloride are commonly stabilized with either 4-methoxyphenol or phenothiazine, in order to prevent polymerization. It is preferred to employ phenothiazine-stabilized acryloyl chloride in the reaction with (It), as it yields a purer crosslinker product.

The hydrochloric acid by-product of the reaction suitably is neutralized or removed by a scavenging agent. Commonly employed scavenging agents are well known to those skilled in the art and include amines, such as triethylamine, trimethylamine and the like. Other basic, i.e. alkaline, scavenging reagents may be used. In the case of an amine, such as triethylamine, a stoichiometric amount of amine is suitably employed, such that an equivalent of amine is used to an equivalent of hydrochloric acid produced in the reaction. In a preferred embodiment of the invention, an excess of amine is utilized in order to achieve improved reaction yields. The amount, if any, of excess scavenging agent is determined empirically based upon the purity of the reagents, solvent and other conditions.

An inert polar or non-polar solvent suitably is employed in the preparation of the crosslinker. Examples of suitable solvents include toluene, dichloromethane, chloroform, and tetrahydrofuran. Combinations of solvents may be used. The amount of solvent is not critical as long as it is sufficient to dissolve the reagents. The concentration of reagents in the reaction mixture preferably is in the range of from about 0.01 Molar to about 10 Molar, and more preferably is from about 0.2 Molar to about 4 Molar.

The reaction of acryloyl chloride or methacryloyl chloride with reagents (I^{†}), (II^{†}), or (III^{†}) is exothermic. Therefore, the reaction temperature preferably is controlled so that the temperature does not reach a point at which thermal polymerization begins. The reaction temperature is not critical so long as the reaction proceeds. Preferably, the temperature of the reaction mixture is from about 15° C. to about 55° C.

Any reaction period can be employed; however, generally effective reaction periods fall in the range of from about 1 hour to about 24 hours. The process is preferentially carried out in the presence of an inert atmosphere, such as nitrogen or argon.

The crude product of the reaction may be recovered and treated by methods known to those skilled in the art, such as those described in the examples, in order to obtain the desired product.

The crosslinker of the invention is polymerizable with the monomers that are commonly employed to make commercial superabsorbent polymers. The crosslinker suitably is used directly in the polymerization reaction in an amount sufficient to yield a processable gel in the reactor. In the present invention, the inventive thermally degradable crosslinker preferably is employed in an amount sufficient to yield a tough, processable gel in the polymerization reactor while at the same time allowing the final product to have a high absorption capacity. The exact amount of polyvinyl crosslinker required to achieve this level of toughness will vary, but in one embodiment of the invention is enough to provide an absorption capacity of the polymer after drying but before heat-treatment of at least 10 g/g, and preferably 55 g/g or less, more preferably 45 g/g or less, and most preferably 35 g/g or less. Preferably, the amount of inventive crosslinker compound employed is in the range of from at least about 100 parts per million (ppm), based on the amount of the polymerizable monomer, to about 50,000 ppm.

Mixtures of crosslinkers can be employed. The total amount of all crosslinkers present is sufficient to provide a polymer with good absorptive capacity, good absorption under load, and a low extractable materials content. In a preferred embodiment of the invention, the crosslinker of the invention is employed with an optional second crosslinker, which is preferably a non-vinyl crosslinker. The total amount of crosslinker employed advantageously is at least about 100 ppm by weight based on the amount of the polymerizable monomer fed to the reactor, preferably is at least about 1,000 ppm, more preferably is at least about 2,000 ppm, and most preferably is at least about 5,000 ppm. Preferably, the total amount of crosslinker employed advantageously is about 50,000 parts per million or less by weight based upon the amount of the polymerizable monomer present, more preferably is about 20,000 ppm or less and most preferably is about 15,000 ppm or less. In various embodiments of the invention, the amount of the inventive crosslinker employed is from about 100 ppm to about 50,000 ppm, from about 500 ppm to about 20,000 ppm, from about 1,000 ppm to about 15,000 ppm, or from about 2,000 ppm to about 10,000 ppm.

The water-absorbent, water-insoluble polymer employed in the invention advantageously is derived from one or more ethylenically unsaturated carboxylic acids, ethylenically unsaturated carboxylic acid anhydrides or salts thereof. The polymer can be prepared using comonomers known in the art for use in preparing superabsorbent polymers including comonomers such as an acrylamide, an acrylonitrile, a vinyl pyrrolidone, a vinyl sulphonic acid or a salt thereof. If employed, the comonomer advantageously can comprise up to 75 percent by weight of the monomer mixture. The polymer optionally can be prepared by grafting the monomer and/or comonomer onto a graft substrate such as a cellulosic polymer, a modified cellulosic polymer, a polyvinyl alcohol or a starch hydrolyzate. If employed, the graft substrate advantageously can comprise up to 25 percent by weight of the monomer mixture.

Preferred unsaturated carboxylic acid and carboxylic acid anhydride monomers include the acrylic acids typified by acrylic acid, methacrylic acid, ethacrylic acid, α-chloroacrylic acid, α-cyano acrylic acid, β-methyl acrylic acid (crotonic acid), α-phenyl acrylic acid, β-acryloyloxy propionic acid, sorbic acid, α-chloro sorbic acid, angelic acid, cinnamic acid, p-chloro cinnamic acid, beta-styrenic acrylic acid (1-carboxy-4-phenyl butadiene-1,3), itaconic acid, citraconic acid, mesaconic acid, glutaconic acid, maleic acid, fumaric acid and maleic acid anhydride. More preferably, the starting monomer is acrylic acid, methacrylic acid, or a salt thereof, with acrylic acid or a salt thereof being most preferred.

Preferably, 25 mole percent or greater of the carboxylic acid units of the hydrophilic polymer are neutralized with base, more preferably 50 percent or greater, and most preferably 65 percent or greater. This neutralization may be performed after completion of the polymerization. In a preferred embodiment, the starting monomer mix has carboxylic acid moieties that are neutralized to the desired level prior to polymerization. The final polymer or the starting monomers may be neutralized by contacting them with a salt-forming cation. Such salt-forming cations include alkali metal, ammonium, substituted ammonium and amine based cations. Preferably, the polymer is neutralized with an alkali metal hydroxide such as, for example, sodium hydroxide or potassium hydroxide, or an alkali metal carbonate or bicarbonate such as, for example, sodium carbonate or potassium carbonate.

The water-absorbent polymers of the invention are crosslinked to make them water-insoluble. Optionally, vinyl, non-vinyl, allylic or dimodal crosslinkers can be employed in various combinations with the crosslinker of the invention. Polyvinyl crosslinkers commonly known in the art for use in superabsorbent polymers advantageously are employed. Preferred compounds having at least two polymerizable double bonds include: polyvinyl compounds such as divinyl benzene, divinyl toluene, divinyl xylene, divinyl ether, divinyl ketone and trivinyl benzene; polyesters of unsaturated mono- or polycarboxylic acids with polyols, such as di- or tri-(meth)acrylic acid esters of polyols such as ethylene glycol, diethylene glycol, triethylene glycol, tetra ethylene glycol, propylene glycol, dipropylene glycol, tripropylene glycol, tetrapropylene glycol, trimethylol propane, glycerin, polyoxyethylene glycols and polyoxypropylene glycols; unsaturated polyesters that can be obtained by reacting any of the above-mentioned polyols with an unsaturated acid such as maleic acid; polyesters of unsaturated mono- or polycarboxylic acids with polyols derived from reaction of C₂-C₁₀ polyhydric alcohols with 2 to 8 C₂-C₄ alkylene oxide units per hydroxyl group, such as trimethylol propane hexaethoxyl triacrylate; di- or tri-(meth)acrylic acid esters that can be obtained by reacting a polyepoxide with (meth)acrylic acid; bis(meth)acrylamides such as N,N-methylene-bisacrylamide; carbamyl esters that can be obtained by reacting polyisocyanates such as tolylene diisocyanate, hexamethylene diisocyanate, 4,4′-diphenyl methane diisocyanate and NCO-containing prepolymers obtained by reacting such diisocyanates with active hydrogen atom-containing compounds with hydroxyl group-containing monomers, such as di-(meth)acrylic acid carbamyl esters obtainable by reacting the above-mentioned diisocyanates with hydroxyethyl(meth)acrylate; poly(meth)allyl ethers of polyols, (including polyols such as alkylene glycols, glycerol, polyalkylene glycols, polyoxyalkylene polyols and carbohydrates) including, for example, polyethylene glycol diallyl ether, allylated starch, and allylated cellulose; poly-allyl esters of polycarboxylic acids, such as diallyl phthalate and diallyl adipate; poly(meth)allyl amines such as diallylamine, triallylamine, tetraallyl alkylene diamines, diallyl dialky ammonium halides, tetraallyl ammonium halides and others; and esters of unsaturated mono- or polycarboxylic acids with mono(meth)allyl ester of polyols, such as allyl methacrylate or (meth)acrylic acid ester of polyethylene glycol monoallyl ether.

The preferred classes of optional crosslinkers include, for example, bis(meth)acrylamides; allyl(meth)acrylates; poly-esters of (meth)acrylic acid with polyols such as diethylene glycol diacrylate, trimethylol propane triacrylate, and polyethylene glycol diacrylate; and polyesters of unsaturated mono- or poly-carboxylic acids with polyols derived from the reaction of C₁-C₁₀ polyhydric alcohols with 2 to 8 C₂-C₄ alkylene oxide units per hydroxyl group, such as ethoxylated trimethylol propane triacrylate. More preferably, the optional crosslinking agent corresponds to Formula IV:

R₄R₅O_{q}—C(O)R₆)_{g} (IV)

wherein:
R₄ is a straight- or branched-chain polyalkoxy radical with 1 to 10 carbon atoms, optionally substituted with one or more oxygen atoms in the backbone, having g valences;
R₅ is independently in each occurrence an alkylene group of 2 to 4 carbon atoms;
R₆ is independently in each occurrence a straight- or branched-chain alkenyl moiety with 2 to 10 carbon atoms;
q is a number from 1 to 20; and
g is a number from 2 to 8.

In the most preferred embodiment the polyvinyl crosslinker corresponds to Formula IV wherein R₄ is derived from trimethylolpropane, R₅ is ethylene —(CH₂CH₂)—, R₆ is vinyl —(CH═CH₂), the average value of q is from 2 to 6, and g is 3. The most preferred polyvinyl crosslinker is highly ethoxylated trimethylolpropane triacrylate, containing an average of 15 to 16 ethoxyl groups per molecule of trimethylolpropane. Crosslinkers corresponding to Formula IV are available from Craynor under the trademark Craynor and from Sartomer under the trademark Sartomer. Generally, the crosslinkers described by Formula IV are found as a mixture of materials described by the formula and by-products resulting from the preparation process. Mixtures of polyvinyl crosslinkers can be employed.

The non-vinyl crosslinkers of this invention are agents having at least two functional groups capable of reacting with the carboxyl groups of the polymer, and include materials such as glycerin, polyglycols, ethylene glycol digylcidyl ether, and diamines. Many examples of these crosslinkers are given in U.S. Pat. Nos. 4,666,983 and 4,734,478, which teach the application of such crosslinkers to the surface of absorbent polymer powder followed by heating to crosslink surface chains and improve absorption capacity and absorption rate. Additional examples are given in U.S. Pat. No. 5,145,906, which teaches post-crosslinking with such crosslinkers. In the present invention, the non-vinyl crosslinkers, if employed, advantageously can be added homogeneously to the polymerization mixture at the start of the process. Preferred non-vinyl crosslinkers include hexane diamine, glycerin, ethylene glycol diglycidyl ether, ethylene glycol diacetate, polyethylene glycol 400, polyethylene glycol 600, and polyethylene glycol 1000. Examples of more preferred non-vinyl crosslinkers include polyethylene glycol 400 and polyethylene glycol 600. Mixtures of non-vinyl crosslinkers can be employed.

The dimodal crosslinkers that can be employed in the process of this invention are compounds that have at least one polymerizable vinyl group and at least one functional group capable of reacting with carboxyl groups. The term “dimodal crosslinkers” is used to distinguish these from normal vinyl crosslinkers, because they use two different reaction modes to form a crosslink. Examples of dimodal crosslinkers include hydroxyethyl methacrylate, polyethylene glycol monomethacrylate, glycidyl methacrylate, and allyl glycidyl ether. Many examples of this type of compound are given in U.S. Pat. Nos. 4,962,172 and 5,147,956, which teach the manufacture of absorbent films and fibers by (1) the preparation of linear copolymers of acrylic acid and hydroxyl containing monomers, (2) forming solutions of these copolymers into the desired shapes, and (3) fixing the shape by heating the polymer to form ester crosslinks between the pendant hydroxyl and carboxyl groups. In the process of the present invention, the dimodal crosslinker, if employed, advantageously is added homogeneously to the polymerization mixture at the start of the process. Preferred dimodal crosslinkers include hydroxyethyl(meth)acrylate, polyethylene glycol 400 monomethacrylate, and glycidyl methacrylate. Hydroxyethyl(meth)acrylate is an example of a more preferred dimodal crosslinker. Mixture of dimodal crosslinkers can be employed.

Polymerization can be accomplished under polymerization conditions in an aqueous or nonaqueous polymerization medium or in a mixed aqueous/nonaqueous polymerization medium. As used herein, the term “aqueous medium” means water, or water in admixture with a water-miscible solvent. Examples of water-miscible solvents include lower alcohols. Preferably the aqueous medium is water. Examples of nonaqueous polymerization media include various inert hydrophobic liquids which are not miscible with water, such as substituted or unsubstituted aromatic or aliphatic hydrocarbons, including halogenated and nonhalogenated liquid hydrocarbons having from about 4 to about 20 carbon atoms per molecule, as well as mixtures of any of the aforementioned media. Conventional additives that are well known in the art, such as surfactants, can be incorporated into the polymerization mixture.

The monomers and crosslinkers are preferably dissolved, dispersed or suspended in a suitable polymerization medium, such as, for example, the aqueous medium, at a concentration level of 15 percent by weight or greater, more preferably 25 percent or greater, and most preferably 29 percent or greater, based on the total weight of the reactor contents. The monomers and crosslinkers are preferably dissolved, dispersed or suspended in the aqueous medium.

Another component used to prepare the superabsorbent polymers is a free radical initiator, which may be any conventional polymerization initiator suitable for use in solution polymerization including, for example, peroxygen compounds such as sodium, potassium and ammonium peroxodisulfates, caprylyl peroxide, benzoyl peroxide, hydrogen peroxide, cumene hydroperoxide, tertiary butyl diperphthalate, tertiary butyl perbenzoate, sodium peracetate and sodium percarbonate. Conventional redox initiator systems, which are formed by combining the foregoing peroxygen compounds with reducing agents, can also be utilized. Examples of reducing agents include sodium bisulfite, sodium thiosulfate, L- or iso-ascorbic acid or a salt thereof, and oxidizable metal salts such as ferrous salts. In addition, water soluble azo-compounds such as 2,2′-azobis(2-amidinopropane hydrochloride) or 4,4′-azobis(4-cyanovaleric acid) and its alkali metal, alkaline earth metal or ammonium salts may be used. As is known in the art, the initiator is used in an amount sufficient to initiate the polymerization. The initiator can be present in an amount of up to 5 mole percent, based on the total moles of polymerizable monomer present. More preferably, the initiator is present in an amount of from 0.001 to 0.5 mole percent based on the total moles of polymerizable monomer in the aqueous medium. Mixtures of initiators can be employed.

It is also possible, as is well-known to those skilled in the art, to prepare the polymer of the current invention with the addition of recycled “fines” to the polymerization mixture, or to the polymer gel following polymerization. The amount of fines added to the polymerization mixture is preferably less than 12 weight percent based on the amount of monomer in the polymerization mixture, more preferably less than 10 weight percent, and most preferably less than 8 weight percent.

As is well-known to those skilled in the art, it is also possible to carry out the polymerization process using multiphase polymerization processing techniques such as inverse emulsion polymerization or inverse suspension polymerization procedures. In the inverse emulsion polymerization or inverse suspension polymerization procedures, the aqueous reaction mixture as hereinbefore described is suspended in the form of tiny droplets in a matrix of a water-immiscible, inert organic solvent such as cyclohexane. Polymerization occurs in the aqueous phase, and suspensions or emulsions of this aqueous phase in an organic solvent permit better control of the exothermic heat of polymerization and further provide the flexibility of adding one or more of the aqueous reaction mixture components in a controlled manner to the organic phase.

When inverse suspension polymerization or inverse emulsion polymerization techniques are employed, additional ingredients such as surfactants, emulsifiers and polymerization stabilizers may be added to the overall polymerization mixture. When any process employing organic solvent is utilized, it is important that the hydrogel-forming polymer material recovered from such processes be treated to remove substantially all of the excess organic solvent. Preferably, the hydrogel-forming polymers contain no more than 0.5 percent by weight of residual organic solvent.

In one embodiment of the invention, at least one chlorine- or bromine-containing oxidizing agent is added to the monomer mixture or to the wet hydrogel according to techniques well-known to those skilled in the art. It is preferably added to the monomer mixture. Preferred oxidizing agents are bromates, chlorates and chlorites. Preferably a chlorate or bromate salt is employed as the oxidizing agent. Chlorine-containing oxidizing agents are preferred. The counterion of the bromate or chlorate salt can be any counterion which does not significantly interfere in the preparation of the polymers or their performance. Preferably, the counterions are alkaline earth metal ions or alkali metal ions. More preferred counterions are the alkali metals, with potassium and sodium being even more preferred.

The chlorine- or bromine-containing oxidizing agent is present in a sufficient amount such that after heat-treatment the desired balance of polymer properties, such as absorption capacity, absorption under load (AUL) and residual monomer, is achieved. Preferably, at least 10 ppm by weight of a chlorine- or bromine-containing oxidizing agent is employed, based on the total weight of monomers, more preferably at least 50 ppm, and even more preferably at least 100 ppm and most preferably at least 200 ppm. Desirably, the amount of a chlorine- or bromine-containing oxidizing agent added is 2000 ppm or less by weight based on the monomers, more desirably 1000 ppm or less, preferably 800 ppm or less and most preferably 500 or less.

The process of the invention may be performed in a batch or continuous manner. The polymerization mixture in the polymerization medium is subjected to polymerization conditions, well-known to those skilled in the art, that are sufficient to produce the water-absorbent polymer.

Preferably, the reaction is performed under an inert gas atmosphere, for example, under nitrogen or argon. The reaction may be performed at any temperature at which polymerization occurs, preferably 0° C. or greater, more preferably 25° C. or greater and most preferably 50° C. or greater. The reaction is conducted for a time sufficient to result in the desired conversion of monomer to crosslinked hydrophilic polymer. Preferably, the conversion is 85 percent or greater, more preferably 95 percent or greater and most preferably 98 percent or greater. Advantageously, initiation of the reaction occurs at a temperature of at least 0° C.

During polymerization, the polymer of the invention generally absorbs all of the aqueous reaction medium to form a hydrogel. The polymer is removed from the reactor in the form of an aqueous hydrogel. The term “hydrogel” as used herein refers to water swollen superabsorbent polymer or polymer particles. In a preferred embodiment, hydrogels coming out of the reactor comprise 15 to 50 percent by weight polymer, with the remainder comprising the polymerization medium and any unreacted components. In a more preferred embodiment the hydrogel comprises 25 to 45 percent polymer. The hydrogel preferably is processed into a particulate shape during the polymerization reaction process in the reactor by the agitator to facilitate the removal of the hydrogel from the reactor. Preferred particle sizes of the hydrogel range from 0.001 to 25 cm, more preferably from 0.05 to 10 cm. In multiphase polymerization, the superabsorbent polymer hydrogel particles may be recovered from the reaction medium by azeotropic distillation and/or filtration followed by drying. If recovered by filtration, then some means of removing the solvents present in the hydrogel must be used. Such means are commonly known in the art.

After removal from the reactor, the hydrogel polymer preferably is subjected to comminution, such as, for example, by a convenient mechanical means of particle size reduction, such as grinding, chopping, cutting or extrusion. The size of the gel particles prior to drying should be such that homogeneous drying of the particles can occur. Preferred particle sizes of the hydrogel range from 0.5 to 3 mm. This particle size reduction can be performed by any means known in the art that gives the desired result. Preferably, the particle size reduction is performed by extruding the hydrogel, optionally followed by chopping of the extrudate.

The comminuted hydrogel polymer particles are subjected to drying conditions to remove the desired amount of the remaining polymerization medium. Desirably, the moisture content of the polymer, after drying to remove the polymerization medium and any dispersing liquid including the optional solvent and the desired amount of the water, is between zero and 20 weight percent, preferably between 5 and 10 weight percent.

The temperature at which the drying takes place is a temperature high enough such that the polymerization medium and liquid including water and optional solvent is removed in a reasonable time period. In a preferred embodiment of the invention, some degradation of the inventive crosslinker will occur during drying. The degree of degradation will depend on the drying time and temperature. Preferably, the drying temperature is 180° C. or less. Desirably, the temperature during drying is 100° C. or above, preferably 120° C. or above and more preferably 130° C. or above. The drying time should be sufficient to remove the desired amount of the water and optional solvent. Preferably, a minimum time for drying is 10 minutes or greater, with 15 minutes or greater being preferred. Preferably, the drying time is 180 minutes or less, with 60 minutes or less being more preferred. In a preferred embodiment, drying is performed under conditions such that water, and optional solvent, volatilizing away from the absorbent polymer particles is removed. This can be achieved by the use of vacuum techniques or by passing inert gases or air over or through the layers of polymer particles. In a preferred embodiment, the drying occurs in dryers in which heated air is blown through or over layers of the polymer particles. Preferred dryers are fluidized beds or belt dryers. Alternatively, a drum dryer may be used. Alternatively, the water may be removed by azeotropic distillation. Such techniques are well known in the art.

During drying, the superabsorbent polymer may form agglomerates and can then be subjected to comminution such as, for example, by mechanical means, to break up the agglomerates. In a preferred embodiment, the superabsorbent polymer is subjected to mechanical particle size reduction means. Such means can include chopping, cutting and/or grinding. The objective is to produce polymer particles having a particle size acceptable for the ultimate end use. In a preferred embodiment, the polymer is chopped and then ground. The final particle size is preferably 2 mm or less, more preferably 0.8 mm or less. Preferably the particles have a size of at least 0.01 mm, more preferably at least 0.05 mm. Particles smaller than this are undesirably small and therefore not suitable for incorporation into personal care articles. These undesirably small particles are commonly referred to as “fines.” The dried superabsorbent polymer particles of the present invention can be used as the basis polymer for further surface crosslinking treatment, for example, by using polyvalent cations like aluminum ions and/or using one of the crosslinkers mentioned above for coating and subsequent heating at elevated temperatures. Surface crosslinking procedures are well known in the art.

In one embodiment of the invention, the polymer particles, optionally coated with surface crosslinking reagents or other substances, are subjected to a heat-treatment step after drying and optional particle size reduction. Heat-treatment of the polymer provides a beneficial increase in the absorption under load (AUL) of the superabsorbent polymer, particularly the AUL under higher pressures. Suitable devices for heat-treatment include, but are not limited to, rotating disc dryers, fluid bed dryers, infrared dryers, agitated trough dryers, paddle dryers, vortex dryers, and disc dryers. One of ordinary skill in the art would vary the time and temperature of heat-treatment as appropriate for the heat transfer properties of the particular equipment used to achieve the desired level of physical properties.

The time period and temperature of the heat-treatment step are chosen such that the absorption properties of the polymer are improved as desired. The polymers are desirably heat-treated at a temperature of 170° C. or above, more desirably 180° C. or above, preferably 200° C. or above, and most preferably 220° C. or above. Preferably, the temperature is 250° C. or below and more preferably 235° C. or below. The polymers are heated to the desired heat-treatment temperature and preferably maintained at such temperature for 1 minute or more and more preferably 5 minutes or more and most preferably 10 minutes or more. If the heating time is too long it becomes uneconomical and there is a risk that the polymer may be damaged. Preferably, polymer particles are maintained at the desired temperature for 60 minutes or less, preferably 40 minutes or less. The properties of the polymer particles can be adjusted and tailored by adjustment of the temperature and the time of the heating step.

After heat-treatment the polymer particles may be difficult to handle due to static electricity. It may be desirable to rehumidify the particles to reduce or eliminate the effect of the static electricity. Methods of humidification of polymer particles are well known in the art. In a preferred mode, the polymer particles are contacted with water and/or water vapor. The polymer particles are contacted with a sufficient amount of water to reduce or eliminate the effects of the static electricity, yet not so much so as to cause the particles to agglomerate. Preferably, the polymer particles are humidified with at least 0.3 parts of water and more preferably at least 5 parts of water, based on 100 weight parts of polymer particles prior to remoisturization. Preferably, the polymer particles are humidified with 10 parts or less by weight of water and more preferably 6 parts or less by weight of water. Optionally, agglomeration prevention or rehydration additives may be added to the crosslinked hydrophilic polymer. Such additives are well known in the art and include surfactants, certain salt solutions, and inert inorganic particles such as silica.

A dust control agent, for example a hydrophobic agent or a hydrophilic agent, such as a propoxylated polyol, can be employed in the preparation of the water-absorbent water-insoluble polymer. The propoxylated polyols are particularly suitable for binding the fine dust of the final superabsorbent polymer particles without causing agglomeration, and for binding the fine particles of powdery additives on the surface. The addition of the propoxylated polyol further results in a more homogeneous distribution of other aqueous additives on the surface of the superabsorbent polymer particles in the absence of organic solvent. Exemplary propoxylated polyols are available from The Dow Chemical Company under the brand name VORANOL. The propoxylated polyol is advantageously used in an amount of from 500 to 2,500 ppm, based on the weight of dry polymer. The concentration of the propoxylated polyol in water preferably ranges from 1 to 10 weight percent and more preferably from 3 to 6 weight percent.

In one embodiment, the dried and optionally heat-treated polymer particles are surface treated with a multivalent metal salt, such as aluminum sulfate. The salt may be added as an aqueous solution, or can be dry blended with the polymer particles with or without a binder. The salt is preferably used in an amount of from 0.1 to 10 weight parts based on 100 parts dry polymer, and desirably has a concentration in water of from 5 to 49 weight percent when employed in a solution.

To increase the flowability of the dried and optionally heat-treated polymer particles, silicon dioxide, preferably fumed silica, or other fine inorganic or organic powders may be mixed with the polymer particles. The optional flowability additive is preferably used in amounts of from 0.01 to 5 weight parts, and more preferably from 0.05 to 3 weight parts, all based on 100 parts dry polymer. An exemplary fumed silica is Aerosil R972, available from Degussa AG, Germany. The additives may be added dry or in dispersed form, such as in the form of an aqueous dispersion.

The polymer of the invention may be in the form of particles or other forms, such as fibers.

The water-absorbent polymers of this invention can be used in any use wherein absorption and binding of aqueous fluids is desired. In a preferred embodiment, the superabsorbent polymer particles of this invention are mixed into or attached to a structure of absorbent material such as synthetic or natural fibers or paper-based woven or nonwoven fibers to form an absorbent structure. In such an absorbent structure the woven or nonwoven structure functions as a mechanism for wicking and transporting fluid via capillary action to the superabsorbent polymer particles which bind and retain such fluids. Examples of such structures are sanitary napkins, diapers, and adult incontinence structures. Other uses of superabsorbent polymers include applications in, for example, medical care, fire fighting, agriculture, horticulture, gardening, pet litter, fertilizer, and packaging, including food packaging.

The absorbent structures according to the present invention comprise means to contain the superabsorbent polymer particles. Any means capable of containing the described superabsorbent polymer particles, which means is further capable of being positioned in a device such as an absorbent garment, is suitable for use in the present invention. Many such containment means are known to those skilled in the art. For example, the containment means may comprise a fibrous matrix such as an airlaid or wetlaid web of cellulosic fibers, a meltblown web of synthetic polymeric fibers, a spunbonded web of synthetic polymeric fibers, a coformed matrix comprising cellulosic fibers and fibers formed from a synthetic polymeric material, airlaid heat-fused webs of synthetic polymeric material or open-celled foams. In one embodiment, it is preferred that the fibrous matrix comprise less than 10, preferably less than 5, weight percent of cellulosic fibers. The containment means may comprise a support structure, such as a polymeric film, on which the superabsorbent polymer particles are affixed. The superabsorbent polymer particles may be affixed to one or both sides of the support structure which may be water-pervious or water-impervious.

The absorbent structures according to the present invention are suited to absorb various fluids including body fluids such as, for example, urine, menses, and blood, and are suitable for use in absorbent garments such as diapers, adult incontinent products and bed pads; in catamenial devices such as sanitary napkins and tampons; and in other absorbent products such as, for example, wipes, bibs and wound dressings. Accordingly, in another aspect, the present invention relates to an absorbent garment comprising an absorbent structure as described above.

Test Methods

### Absorption Capacity (AC)

The absorption capacity is measured according to the method stated in Buchholz, F. L. and Graham, A. T., “Modern Superabsorbent Polymer Technology,” John Wiley & Sons (1998), page 153.

### Absorption Under Load

The absorption under load is measured according to the method stated in Buchholz, F. L. and Graham, A. T., “Modern Superabsorbent Polymer Technology,” John Wiley & Sons (1998), page 160.

### Extractables

One gram of water-absorbent resin particles and 185 mL of 0.9 percent saline solution are placed in a 250 mL jar which is capped and put on a shaker for 16 hours. A part of the extraction solution is filtered. With the aid of a Metrohm Titroprocessor, the pH of a defined volume of the filtrate is adjusted to pH 10 by 0.1 N NaOH, and is finally titrated to pH 2.7 by 0.1 N hydrochloric acid, to determine the amount of extractable materials which are in the filtrate.

The following examples are provided to illustrate the invention and are not intended to limit the scope of the claims. All parts and percentages are by weight unless otherwise indicated.

### SPECIFIC EMBODIMENTS OF THE INVENTION

### Example 1

A 1 L jacketed, bottom drain, reactor is equipped with a nitrogen inlet, thermowell, pitched blade turbine type agitator and addition funnel. The apparatus is purged with nitrogen overnight prior to use. The reactor is charged with 200 ml of toluene and 28.3 g (0.27 mole) of 3-methyl-1,3-butanediol. With stirring, 84.3 g (0.83 mole) of triethylamine are added forming a clear, colorless solution with no increase in temperature. The solution is heated to 35° C. A solution of 109.4 g (1.21 mole) of 96% acryloyl chloride in 100 ml of toluene is added dropwise. A precipitate immediately forms and the reaction temperature increases. The reaction temperature is maintained between 45° C. and 50° C. by jacket cooling. Upon completion of the addition, the mixture is heated at 48° C. for 3 hours.

The mixture is cooled to 35° C. and 500 ml of deionized water are added. The mixture is stirred at 35° C. for 45 minutes in order to dissolve the precipitate. The phases are allowed to settle and the aqueous phase is removed. The organic phase is washed with dilute aqueous sodium chloride in order to facilitate phase separation. The organic phase is isolated and the volatiles removed using a rotary evaporator. The resulting yellowish liquid is pumped with a mechanical vacuum pump at room temperature for 4 hours. The product yield is 40.1 g. 1H and 13C NMR spectra are consistent with 3-methyl-1,3-butanediol diacrylate.

### Example 2

A 500 ml 3-neck round bottom flask is equipped with a nitrogen inlet, magnetic stir bar, additional funnel, temperature probe and stoppers. The flask is charged with 150 ml of toluene and 28.3 ml (0.30 mole) of 97% acryloyl chloride. Via a syringe, 10.6 ml (0.10 mole) of 3-methyl-1,3-butandediol is added. The resulting solution is warmed to 40° C. A solution of 30.6 ml (0.22 mole) of triethylamine in 100 ml of toluene is added at a slow dropwise rate with vigorous stirring. The reaction is exothermic, with a temperature rise to 50° C. The reaction temperature is maintained at approximately 50° C. by cooling with a water bath. During the addition, a flocculent precipitate forms. Upon completion of the addition, the slurry is held at approximately 50° C. via a water bath for 2 hours. After cooling to ambient temperature, the precipitate is removed by filtration. The volatiles are removed from the filtrate under vacuum leaving a pale yellow, somewhat cloudy liquid. This product is dissolved in 80 ml of hexane/toluene (1:1, v:v) and eluted through a column of alumina. The volatiles are removed from the eluent under vacuum, leaving 9.2 g of a clear, very pale yellow liquid. ¹H and ¹³C NMR spectra are consistent with the structure of the desired crosslinker 3-methyl-1,3-butanediol diacrylate.

### Example 3

The crosslinker prepared in Example 2 is employed in a polymerization of partially neutralized acrylic acid as follows.

Samples are prepared in a reactor with a 2 L glass resin kettle bottom, a stainless steel agitator assembly, and a high-torque stirring motor with gear reducers. The kettle bottom has a glass jacket to allow for heating or cooling of the contents using a separate water-circulating temperature bath. The reactor can be sealed with an O-ring that fits into grooves in the kettle bottom and the steel agitator top. The monomer mix is prepared by adding 328.49 g of acrylic acid to a beaker, followed by water (377.02 g) Versenex®80 (trademark of The Dow Chemical Company) chelating agent (0.41 g), vinyl crosslinker, and optionally non-vinyl or dimodal crosslinker. To this mixture is added, with stirring, a solution of 157.03 g of sodium carbonate in 392.57 g of water. The monomer mix is loaded to the reactor under vacuum via a loading tube and the mixture is sparged with nitrogen for 1 hour to remove dissolved oxygen. Next in sequence, 10 percent sodium persulfate in water (7.88 g) and 10 percent sodium erythorbate in water (0.72 g) is added via syringe. The exothermic reaction typically reaches a peak temperature of about 85° C. after about 30 minutes at which point the vessel is heated to 65° C. for 3 hours.

The resulting polymer gel crumb is dried at 100° C. for 16 hours in a forced air oven and then is ground. The resulting particles are sieved to obtain a 30/50 mesh fraction and are heat treated in an oven for one hour at the temperatures indicated in Table 1. The usefulness of this new crosslinker is indicated by the data in Table 1. The initially prepared product, indicated as the control in the table, has a saline absorption capacity of approximately 27 g/g. By thermal treatment at 175° C. to 210° C. for one hour, the capacity of the initial product significantly increases as the heat treatment temperature increases. For a superabsorbent polymer, this controllable increase in AC is a desirable property that is often difficult to achieve in a convenient manufacturing process.

TABLE 1
Capacity with Thermal Treatment*

| | Heat Treatment | |
|---|---|---|
| | Temperature (° C.) | |
| Sample | for 1 hour | AC (g/g) |
| 1 | Control | 27 |
| 2 | 175. | 32 |
| 3 | 185. | 45 |
| 4 | 195. | 52 |
| 5 | 210. | 65 |

*Heat treatment is carried out on 2 gram (30/50 mesh) portions.

### Example 4

The polymerization procedure of Example 3 is repeated with the following exceptions. PEG 600, a polyethylene glycol with a Mn of approximately 600, and glycerin are added to the feed mixture in the amounts shown in Table 2, as are 200 ppm of sodium chlorate. In this example, the products, after drying and grinding, are heat treated in a fluidized bed. Once the fluidized bed heat treater reaches the desired target temperature, approximately 50 g of polymer sample are placed in the zone and a contact thermometer is placed in the sample. The temperature of the sample is monitored until it stabilizes at the target temperature. The superabsorbent polymer particles are heat treated at 230° C. for 20 minutes. The AC and 0.9 psi AUL results are shown in Table 2. Thus, utilizing 3-methyl-1,3-butanediol diacrylate (MBDDA), in combination with PEG 600 and glycerin, it is possible to conveniently produce a superabsorbent polymer with high AC and high AUL.

TABLE 2
Properties of SAPs Produced with MBDDA, PEG 600 and Glycerin.
Crosslinker

| | MBDDA | PEG 600 | Glycerin | AC | 0.9 psi AUL |
|---|---|---|---|---|---|
| Example | (ppm) | (ppm) | (ppm) | g/g | g/g |
| 4-1 | | | | | |
| 4-2 | 6000 | 6000 | 200 | 41.7 | 17.2 |
| 4-3 | 6000 | 6000 | 400 | 33.6 | 25.2 |
| 4-4 | 6000 | 3000 | 200 | 46.3 | 15.9 |
| 4-5 | 6000 | 3000 | 400 | 33.7 | 25.8 |

1. A crosslinker composition comprising a compound of at least one of the following formulas: 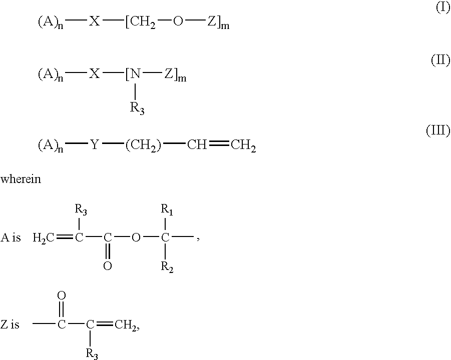
X is an aromatic moiety, an aliphatic moiety, or a mixture thereof, Y is O, N, an aliphatic moiety that may contain one or more O or N atoms, or a mixture thereof, n is from 1 to about 3, m is from 1 to about 3, R₁ and R₂ are independently C₁ to C₄ alkyl, and each R₃ is independently H or methyl.

2. The crosslinker composition of claim 1 wherein the majority of the mass of the composition comprises a compound of Formula I, II, III or a mixture thereof.

3. The crosslinker composition of claim 1 wherein 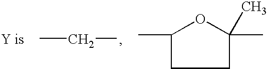
or a mixture thereof.

4. The crosslinker composition of claim 1 wherein X is —CH₂.

5. The crosslinker composition of claim 1 wherein R₁ and R₂ are methyl.

6. The crosslinker compound of claim 1 of the formula:
A-X—CH₂O-Z, wherein R₃ is independently H or methyl, R₁ and R₂ are independently C₁ to C₄ alkyl, X is an aliphatic that contains one or more —O— or —N— atoms, or a mixture thereof.

7. The crosslinker compound of claim 1 of the formula: 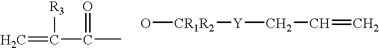
each R₁ and R₂ is independently a C₁-C₄ alkyl moiety; R₃ is H or methyl; and Y is —CH₂— or

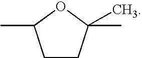

8. The crosslinker compound of claim 1 of the formula:

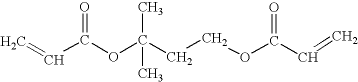

9-10. (canceled)

11. The crosslinker composition of claim 1 wherein the crosslinker composition is 3-methyl-1,3-butanediol diacrylate.

12. A superabsorbent polymer comprising a crosslinker composition comprising a compound of at least one of the following formulas: 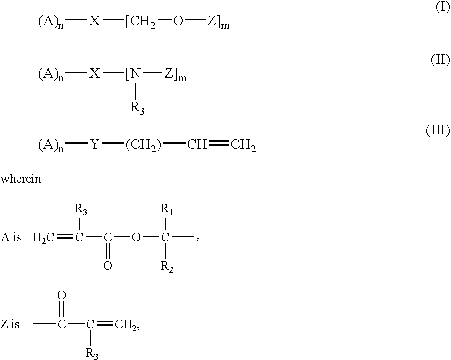
X is an aromatic moiety, an aliphatic moiety, or a mixture thereof, Y is O, N, an aliphatic moiety that may contain one or more O or N atoms, or a mixture thereof, n is from 1 to about 3, m is from 1 to about 3, R₁ and R₂ are independently C₁ to C₄ alkyl, and each R₃ is independently H or methyl.

13. The superabsorbent polymer of claim 12 wherein 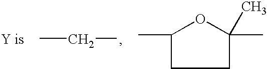
or a mixture thereof.

14. The superabsorbent polymer of claim 12 wherein X is —CH₂.

15. The superabsorbent polymer of claim 12 wherein R₁ and R₂ are methyl.

16. The superabsorbent polymer of claim 12 wherein the formula is

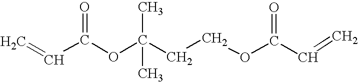

17. The superabsorbent polymer of claim 12 wherein the crosslinker composition is 3-methyl-1,3-butanediol diacrylate.

18. A superabsorbent polymer composition comprising a superabsorbent polymer comprising:
a) at least one monomer selected from an ethylenically unsaturated carboxylic acid, ethylenically unsaturated carboxylic acid anhydride, salts or derivatives thereof based on the superabsorbent polymer; and
b) from about 100 ppm to about 50,000 ppm of the crosslinking composition, based on the monomer of a) wherein the crosslinking composition comprises a compound of at least one of the following formulas: 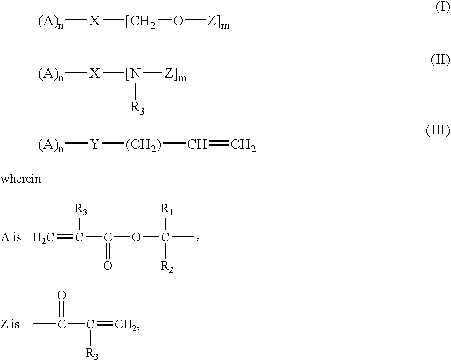
X is an aromatic moiety, an aliphatic moiety, or a mixture thereof, Y is O, N, an aliphatic moiety that may contain one or more O or N atoms, or a mixture thereof, n is from 1 to about 3, m is from 1 to about 3, R₁ and R₂ are independently C₁ to C₄ alkyl, and each R₃ is independently H or methyl;
c) a salt forming cation wherein the superabsorbent polymer has a degree of neutralization of greater than about 25%;
wherein elements a) b) and c) are polymerized into a crosslinked hydrogel, which is then prepared into superabsorbent polymer particles; and the superabsorbent polymer composition further comprises a surface crosslinking agent.

19. The superabsorbent polymer composition of claim 18 wherein 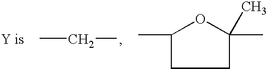
or a mixture thereof.

20. The superabsorbent polymer composition of claim 18 wherein X is —CH₂.

21. The superabsorbent polymer composition of claim 18 wherein R₁ and R₂ are methyl.

22. The superabsorbent polymer composition of claim 18 wherein the formula is

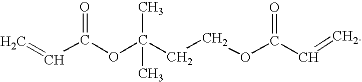

23. The superabsorber polymer composition of claim 18 wherein the crosslinker composition is 3-methyl-1,3-butanediol diacrylate.

24. A diaper having a core, said core comprising at least 10% by weight of the superabsorbent polymer of claim 18.

25. A method to make a superabsorbent polymer comprising the steps of:
a) preparing a superabsorbent polymer by the process of polymerizing of at least one monomer selected from an ethylenically unsaturated carboxylic acid, ethylenically unsaturated carboxylic acid anhydride, salts or derivatives thereof based on the superabsorbent polymer, and from about 100 ppm to about 50,000 ppm of the crosslinking composition comprising a compound of at least one of the following formulas: 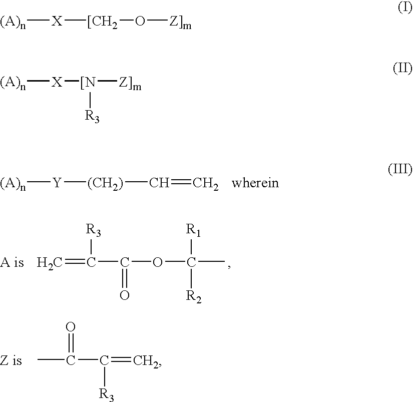
X is an aromatic moiety, an aliphatic moiety, or a mixture thereof, Y is O, N, an aliphatic moiety that may contain one or more O or N atoms, or a mixture thereof, n is from 1 to about 3, m is from 1 to about 3, R₁ and R₂ are independently C₁ to C₄ alkyl, and each R₃ is independently H or methyl, based on the monomer, and a salt forming cation wherein the superabsorbent polymer has a degree of neutralization of greater than about 25%;
b) polymerizing the components of a) into a hydrogel;
c) preparing superabsorbent polymer particles from the superabsorbent polymer;
d) treating the superabsorbent polymer particles with surface additives including a surface crosslinking agent based on the superabsorbent polymer composition.

26. The process of claim 25 wherein 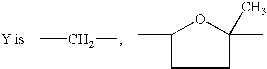
or a mixture thereof.

27. The process of claim 25 wherein X is —CH₂.

28. The process of claim 25 wherein R₁ and R₂ are methyl.

29. The process of claim 25 wherein the formula is

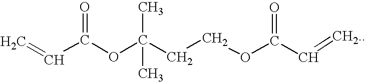

30. The process of claim 25 wherein the crosslinker composition is 3-methyl-1,3-butanediol diacrylate.
